(19) United States
(12) Patent Application Publication
(10) Pub. No.: US 2023/0127330 A1
(43) Pub. Date: Apr. 27, 2023

(54) ATTACHABLE ORTHODONTIC DEVICE CASE

(71) Applicant: Smile Safes, Inc., Hollywood, FL (US)

(72) Inventors: Amnon Arama, Fort Lauderdale, FL (US); Steven Stanimirovic, Holllywood, FL (US)

(21) Appl. No.: 17/511,057

(22) Filed: Oct. 26, 2021

(51) Int. Cl. A61C 19/02; A45D 44/20; A61L 2/10

(52) U.S. Cl. CPC A61C 19/02; A45D 44/20; A61L 2/10; A45D 2200/05; A45D 2200/205; A61L 2202/20; A61L 2202/16; A61L 2202/123; A61L 2202/11

(57) ABSTRACT

A an attachable orthodontic device case includes an orthodontic device case having an interior volume configured for holding an orthodontic device, the orthodontic device case comprising a first half and the second half, wherein the first half is hingably coupled to the second half via a plurality of barrel hinges, a first handle located on the first half of the orthodontic device case, and a male protrusion located on the first handle, a second handle located on the second half of the orthodontic device case, and a female orifice located on the second handle, wherein the female orifice is configured to mate securely with the male protrusion located on the first handle, and, a flat surface on a rear of the second half of the orthodontic device case and a magnet and/or an adhesive on said flat surface for removably coupling to a rear of a mobile computing device.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention disclosed broadly relates to the field of orthodontic devices, and more particularly relates to the field of cases for storing or holding orthodontic devices.

### BACKGROUND OF THE INVENTION

Orthodontic devices, such as retainers and dental aligners, are costly to the average consumer, ranging in the hundreds to thousands of dollars. This financial burden may preclude those of modest means from getting the orthodontic care that they need, leaving the children of these families to either seek orthodontic care in adulthood or to persist in living with their orthodontic issues. Resolving orthodontic issues in adulthood is even more costly, as the teeth are less malleable than in childhood.

Despite how costly they are, orthodontic devices are often lost or misplaced due to their small size and the tendency of consumers to take them off for activities such as eating, brushing their teeth, or chewing gum. When lost or misplaced, these orthodontic devices are costly to replace. Should the consumer fail to replace them, their teeth will persist in appearing improperly aligned. In hopes of preventing this scenario, consumers may store their orthodontic devices in their pocket or purse, which is impractical and unsanitary. It is impractical to store an orthodontic device in a consumer's pocket or purse due to the risk of the orthodontic device breaking or bending, defeating the purpose of using the orthodontic device to begin with. It is unsanitary to store an orthodontic device in a consumer's pocket or purse due to the risk of the orthodontic device brushing against other items that have not been sanitized, the orthodontic device accumulating bacteria, then the orthodontic device being inserted into the consumer's mouth where the bacteria is then ingested and may put the consumer at risk for disease.

Additionally, today consumers must carry many items on a daily basis. Cellular phones, for example, have become ubiquitous and are typically brought any place a consumer travels, from trips to school and work, as well as movements around the consumers own home. When one considers that consumers must also carry other items on a daily basis, such as keys, wallets, and purses, it can be rather unwieldly for a consumer to carry so many items on a daily basis. The typical consumer simply does not have enough hands or pockets to carry all such items on a daily basis.

Therefore, a need exists to overcome the problems with the prior art as discussed above, and particularly for a more effective and efficient manner of storing and holding orthodontic devices, as well as other items.

### SUMMARY OF THE INVENTION

Briefly, according to one embodiment, an attachable orthodontic device case includes an orthodontic device case having an interior volume configured for holding an orthodontic device, the orthodontic device case comprising a first half and the second half, wherein the first half is hingably coupled to the second half via a plurality of barrel hinges, a first handle located on the first half of the orthodontic device case, and a male protrusion located on the first handle, a second handle located on the second half of the orthodontic device case, and a female orifice located on the second handle, wherein the female orifice is configured to mate securely with the male protrusion located on the first handle, and, a flat surface on a rear of the second half of the orthodontic device case and a magnet and/or an adhesive on said flat surface for removably coupling to a rear of a mobile computing device.

The foregoing and other features and advantages of the present invention will be apparent from the following more particular description of the preferred embodiments of the invention, as illustrated in the accompanying drawings.

### BRIEF DESCRIPTION OF THE DRAWINGS

The accompanying drawings, which are incorporated in and constitute a part of this disclosure, illustrate various example embodiments. In the drawings:

FIG. 1 is a top perspective view of an attachable orthodontic device case in semi-open orientation and separated from a mobile phone, in accordance with one embodiment.

FIG. 2 is another top perspective view of the attachable orthodontic device case in fully open orientation and attached to a mobile phone, in accordance with one embodiment.

FIG. 3 is a top view of the attachable orthodontic device case in closed orientation and attached to the mobile phone, in accordance with one embodiment.

FIG. 4 is a top perspective view of an attachable orthodontic device case in semi-open orientation and separated from a mobile phone, in accordance with a second embodiment.

FIG. 5 is a top perspective view of an attachable orthodontic device case in semi-open orientation and separated from a mobile phone, in accordance with a third embodiment.

### DETAILED DESCRIPTION

The following detailed description refers to the accompanying drawings. Wherever possible, the same reference numbers are used in the drawings and the following description to refer to the same or similar elements. While embodiments may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves over the prior art by providing an improved attachable orthodontic device case that is simple, inexpensive to manufacture and lightweight. The claimed subject matter addresses problems with prior art by designating a proper storage unit adhered to the back of a cellular device for easy storage and effortless mobility. The orthodontic device, to be stored properly and hygienically, should have a designated storage unit that will preserve its shape and protect the consumer from ingesting harmful bacteria. The claimed subject matter addresses concerns regarding hygiene and preserving the structural integrity of the orthodontic device by preventing the orthodontic device from brushing against items that may compromise the shape of the orthodontic device, or when the consumer inserts the device, the consumer's health. By preserving the structural integrity of the orthodontic device, consumers expend less funds on replacing said devices. In this respect, the claimed subject matter is an investment, preventing the unnecessary expenditure of replacing the same device multiple times.

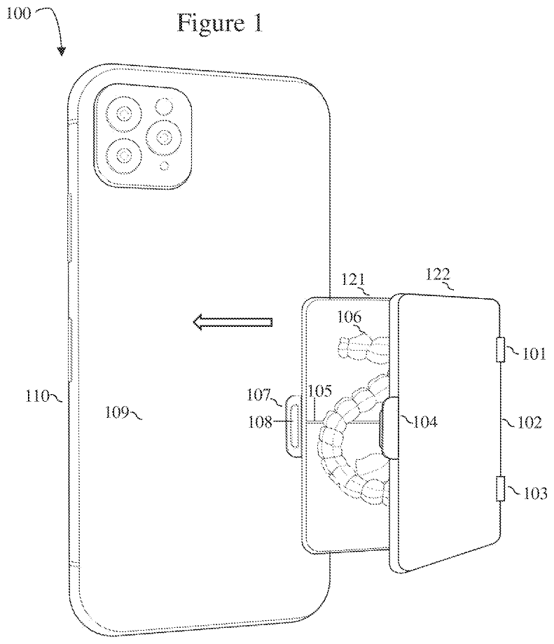

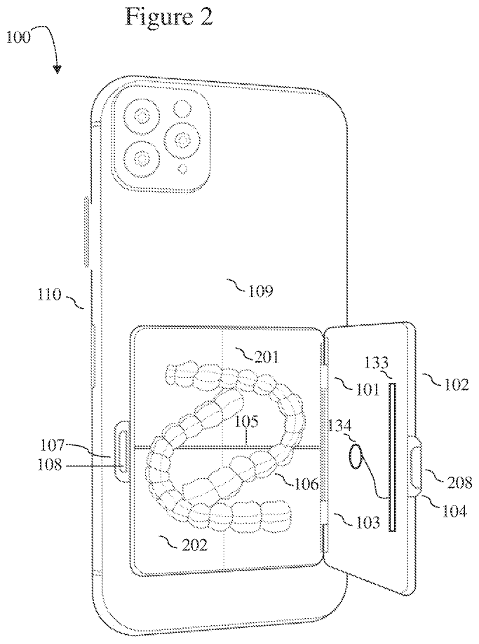

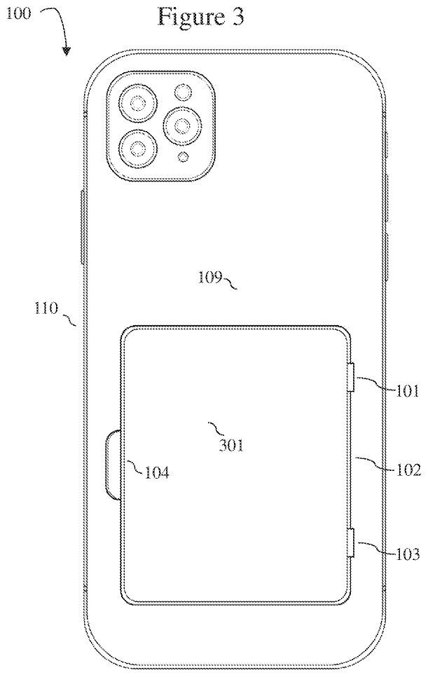

The claimed subject matter will now be described with reference to the FIGS. 1-3. FIG. 1 is a top perspective view of an attachable orthodontic device case 102 in semi-open orientation and separated from a mobile phone 110, in accordance with one embodiment. FIG. 2 is another top perspective view of the attachable orthodontic device case 102 in fully open orientation and attached to a mobile phone 110, in accordance with one embodiment. FIG. 3 is a top view of the attachable orthodontic device case 102 in closed orientation and attached to the mobile phone 110, in accordance with one embodiment. FIGS. 1-3 show a system 100 for combining an attachable orthodontic device case 102 to a mobile phone 110.

FIG. 1 demonstrates orthodontic device 106, more specifically clear aligners, comprised of transparent plastic intended to mold teeth from their current form to the form agreed upon by the patient and the dentist or orthodontist. The clear aligners are created with a mold taken either by a dentist, an orthodontist, or the patient themselves, that is then input into a digital model, referred to as CAD-CAM (computer-aided-design and computer-aided-manufacturing), that determines the exact measurements necessary to mold the teeth from their present state to their desired state. The specific type of plastic used for these clear aligners are medical grade polyurethane resins, which are comprised of both methylene diphenyl diisocyanate and 1.6-hexanediol. Orthodontic device 106 may comprise to distinct and separate pieces—one for the top set of teeth and the other for the bottom set of teeth.

FIG. 1 also demonstrates attachable orthodontic device case 102, which may be comprised of any of the types of plastic well known in the art for said purposes, including polyethylene terephthalate, high-density polyethylene, polyvinyl chloride, polyvinyl chloride, low-density polyethylene, polypropylene, or polystyrene. The case 102 is shaped such that orthodontic device 106 may fit easily within the case without being damaged. The orthodontic device case 102 may be shaped like a rectangular cuboid with rounded edges. The depth of the case 102 is specifically configured, shaped and designed to accommodate orthodontic device 106 without imposing on the consumer's comfort, should the consumer insert the case atop their mobile phone 110 within their pocket.

FIG. 1 demonstrates barrel hinges 101 and 103 that are used to hingably attach the top of the case 122 (or first half of the case) to the bottom of the case 121 (or second half of the case). A barrel hinge is a hinge that opens fully to 180 degrees. Barrel hinges 101 and 103 consist of a barrel, or a hollow cylinder, into which a rod or pivot is inserted such that the barrel acts as a pivot point. The barrel may rotate about the pivot point, such that top of the case 122 rotates about the hingers to allow access to the contents within the bottom of the case 121, which would be an orthodontic device 106. The barrel may be located on the first half of the orthodontic device case, and the rod may be located on the second half of the orthodontic device case. Note the top of the case 122 (or first half of the case) comprises an opening that allows for the insertion of the orthodontic device 106 into the interior volume of the top of the case 122. In one embodiment, the opening may be located at the upper portion of the top of the case 122, at the bottom portion of the top of the case 122, or at the side portion of the top of the case 122.

Handles 104 and 107 (attached to the top 122 and bottom 121 of the case 102, respectively) act in concert to fasten the bottom 121 and top 122 of the case 102 together and protect orthodontic device 106. As demonstrated in FIG. 2, handle 104 comprises a plastic protrusion 208 that fits securely into a gap 108 in the first handle 107, so as to create an air-tight fit, or a near air-tight fit, that withstands minor blows and handling. This fastening ensures that orthodontic device 106 is not accidentally released from case 102 due to the consumer's mishandling of the phone 110 and/or case 102.

Divider 105, as demonstrated in FIG. 2, acts to separate the two pieces of orthodontic device 106 should the consumer choose to place each piece of orthodontic device 106 within the confines created by the divider. Should the consumer choose to use divider 105, the shape of orthodontic device 106 is better preserved, as there is less friction between each piece of orthodontic device 106 as the consumer moves case 102 and/or phone 110 in such a way that may cause the pieces to rub against each other.

Phone 110, as demonstrated by FIG. 1, is a cellular device through which a consumer may call, send text messages, and access cellular data or the Internet through WiFi. Phone 110 may be of any brand and should be adequately sized to fit comfortably in the consumer's hands, pocket, or purse. The surface on the rear of phone 110, which has been labeled in FIG. 1 with item number 109, is flat enough that case 102 may be comfortably affixed without any adjustments to the shape of case 102.

As demonstrated in FIG. 2, a planar surface (on the rear) of the bottom 121 of the case 102 may be deposited with adhesive configured for removably coupling to a flat surface, such as the rear surface 109 of the mobile phone 110. In one alternative to the adhesive, a magnetic element, described more fully herein, may be used to removably couple the case 102 to the mobile phone 110. Note that although the Figures show the case 102 removably coupled to a mobile phone, the claimed subject matter supports the case 102 coupled to any flat surface, including the flat surface of any mobile computing device, any apparatus or any piece of furniture.

In one embodiment, the adhesive may comprise a release coating and adhesive. The release coating may be a polyvinyl carbamate that prevents the adhesive from adhering to itself when pulled off a roll. The case itself may be covered with said release coating. The main component of the adhesive may be a polymer, such as a styrenic block copolymers, water-based acrylics, or silicone adhesives. The adhesive may include other chemicals added to enhance the main polymer's deformability and flow properties. For example, a polyterpene resin may be added to maintain the cross-links that link the polymer's strands but minimizes strand entanglement. This addition increases the polymer's softness and flow.

An interior surface of the top 122 of the case 102 may include a light 133, such as an ultraviolet light, that serves to irradiate the orthodontic device 106 for sanitation purposes when the case 102 is closed. Interior 202 of the case 102 may be bathed in ultraviolet light when the case 102 is closed, resulting in the elimination or reduction of bacteria located within the interior of the case or the orthodontic device 106 or both. The light 133 may be supplied by a small, changeable battery 134. The light 133 may be activated for a set period of time (such as one hour) when the case 102 is closed and deactivated when the case 102 is open.

Interior 202 of the case 102 may be comprised of one of the plastics described in the description for case 102. Interior 202 of the case is expected to be kept sanitary so as to not spread infection or disease as the consumer inserts orthodontic device 106 into his or her mouth. Vertical line 201 within the interior 202 of the case 102 acts similarly to divider 105, as demonstrated in FIG. 2, to allow the consumer to separate each piece of the orthodontic device 106 to preserve the shape of orthodontic device 106 by minimizing friction between the two pieces.

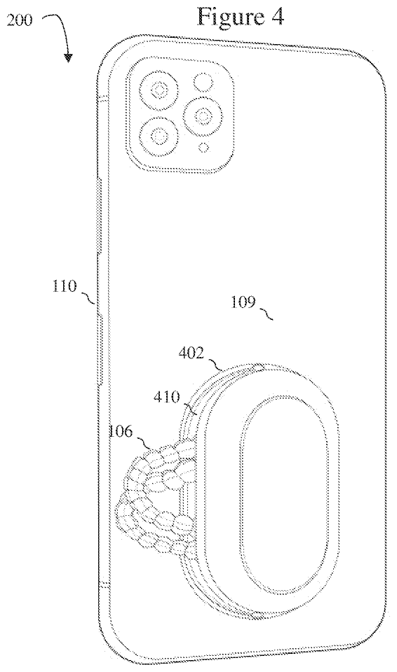

FIG. 4 is a top perspective view of an attachable orthodontic device case 402 in semi-open orientation and attached to a mobile phone 110, in accordance with a second embodiment. FIG. 4 shows a system 200 for combining an attachable orthodontic device case 402 to a mobile phone 110. In this embodiment, the case 402 comprises an oval like shape with an inner volume configured for holding the orthodontic device 106. The case 402 includes an opening 410 on the left side of the case. The case 402 is shaped such that orthodontic device 106 may fit easily within the case without being damaged. The depth of the case 402 is specifically configured, shaped and designed to accommodate orthodontic device 106 without imposing on the consumer's comfort, should the consumer insert the case atop their mobile phone 110 within their pocket.

FIG. 4 demonstrates the case 402 has a top (or first half of the case) and a bottom (or second half of the case). Note the top of the case 402 (or first half of the case) may be manually separated from the bottom (or second half of the case) to create the opening 410 that allows for the insertion of the orthodontic device 106 into the interior volume of the case. Once the case is released, it returns to its original configuration and the opening is closed, sealing in the orthodontic device. In one embodiment, the opening may be located at the side portion of the top of the case 402.

As demonstrated in FIG. 4, a planar surface (on the rear) of the bottom of the case 402 may be deposited with adhesive configured for removably coupling to a flat surface, such as the rear surface 109 of the mobile phone 110. In one alternative to the adhesive, a magnetic element, described more fully herein, may be used to removably couple the case 402 to the mobile phone 110.

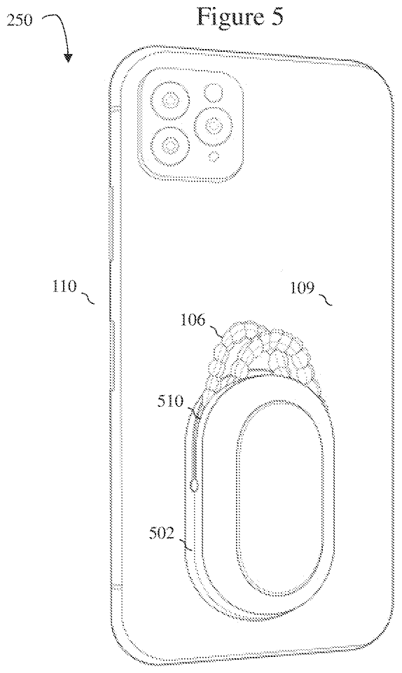

FIG. 5 is a top perspective view of an attachable orthodontic device case 502 in semi-open orientation and attached to a mobile phone 110, in accordance with a third embodiment. FIG. 5 shows a system 250 for combining an attachable orthodontic device case 502 to a mobile phone 110. In this embodiment, the case 502 comprises an oval like shape with an inner volume configured for holding the orthodontic device 106. The case 402 includes an opening 510 on the upper portion of the case. The case 502 is shaped such that orthodontic device 106 may fit easily within the case without being damaged. The depth of the case 502 is specifically configured, shaped and designed to accommodate orthodontic device 106 without imposing on the consumer's comfort, should the consumer insert the case atop their mobile phone 110 within their pocket.

FIG. 5 demonstrates the case 502 has a top (or first half of the case) and a bottom (or second half of the case). Note the top of the case 502 (or first half of the case) may be manually separated from the bottom (or second half of the case) to create the opening 510 that allows for the insertion of the orthodontic device 106 into the interior volume of the case. Once the case is released, it returns to its original configuration and the opening is closed, sealing in the orthodontic device. In one embodiment, the opening may be located at the top portion of the top of the case 502.

As demonstrated in FIG. 5, a planar surface (on the rear) of the bottom of the case 502 may be deposited with adhesive configured for removably coupling to a flat surface, such as the rear surface 109 of the mobile phone 110. In one alternative to the adhesive, a magnetic element, described more fully herein, may be used to removably couple the case 502 to the mobile phone 110.

Although specific embodiments of the claimed embodiments have been disclosed, those having ordinary skill in the art will understand that changes can be made to the specific embodiments without departing from the spirit and scope of the claimed embodiments. The scope of the claimed embodiments is not to be restricted, therefore, to the specific embodiments. Furthermore, it is intended that the appended claims cover any and all such applications, modifications, and embodiments within the scope of the claimed embodiments.

Embodiments herein, for example, are described above with reference to block diagrams and/or operational illustrations of methods and systems, according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is

1. An attachable orthodontic device case comprising:
an orthodontic device case having an interior volume configured for holding an orthodontic device;
the orthodontic device case comprising a first half and the second half, wherein the first half is hingably coupled to the second half via a plurality of barrel hinges;
a first handle located on the first half of the orthodontic device case, and a male protrusion located on the first handle;
a second handle located on the second half of the orthodontic device case, and a female orifice located on the second handle, wherein the female orifice is configured to mate securely with the male protrusion located on the first handle; and
a flat surface on a rear of the second half of the orthodontic device case and an adhesive on said flat surface for coupling to a rear of a mobile computing device.

2. The attachable orthodontic device case of claim 1, wherein the orthodontic device case is shaped like a rectangular cuboid.

3. The attachable orthodontic device case of claim 2, wherein the rectangular cuboid shape of the orthodontic device case includes rounded edges.

4. The attachable orthodontic device case of claim 3, wherein an interior of the orthodontic device case includes a divider for separating different pieces of the orthodontic device.

5. The attachable orthodontic device case of claim 4, wherein each of the plurality of barrel hinges comprises a barrel located on the first half of the orthodontic device case, and a rod located on the second half of the orthodontic device case, such that the rod is inserted into the barrel.

6. The attachable orthodontic device case of claim 5, wherein the orthodontic device case is composed of any one of plastic or a plastic derivative.

7. The attachable orthodontic device case of claim 6, wherein the orthodontic device case is composed of a disposable material.

8. The attachable orthodontic device case of claim 7, further comprising:
an ultraviolet light located within the interior volume of the orthodontic device case, wherein said light is activated when the case is closed.

9. An attachable orthodontic device case comprising:
an orthodontic device case having an interior volume configured for holding an orthodontic device;
the orthodontic device case comprising a first half and the second half, wherein the first half is hingably coupled to the second half via a plurality of barrel hinges;
a first handle located on the first half of the orthodontic device case, and a male protrusion located on the first handle;
a second handle located on the second half of the orthodontic device case, and a female orifice located on the second handle, wherein the female orifice is configured to mate securely with the male protrusion located on the first handle; and
a flat surface on a rear of the second half of the orthodontic device case and a magnet on said flat surface for coupling to a rear of a mobile computing device.

10. The attachable orthodontic device case of claim 9, wherein the orthodontic device case is shaped like a rectangular cuboid.

11. The attachable orthodontic device case of claim 10, wherein the rectangular cuboid shape of the orthodontic device case includes rounded edges.

12. The attachable orthodontic device case of claim 11, wherein an interior of the orthodontic device case includes a divider for separating different pieces of the orthodontic device.

13. The attachable orthodontic device case of claim 12, wherein each of the plurality of barrel hinges comprises a barrel located on the first half of the orthodontic device case, and a rod located on the second half of the orthodontic device case, such that the rod is inserted into the barrel.

14. The attachable orthodontic device case of claim 13, wherein the orthodontic device case is composed of any one of plastic or a plastic derivative.

15. The attachable orthodontic device case of claim 14, wherein the orthodontic device case is composed of a disposable material.

16. The attachable orthodontic device case of claim 15, further comprising:
an ultraviolet light located within the interior volume of the orthodontic device case, wherein said light is activated when the case is closed.

17. An attachable orthodontic device case comprising:
an orthodontic device case having an interior volume configured for holding an orthodontic device;
the orthodontic device case comprising an opening configured for insertion of the orthodontic device; and
a flat surface on a rear of the orthodontic device case and an adhesive on said flat surface for coupling to a rear of a mobile computing device.

18. The attachable orthodontic device case of claim 17, wherein an interior of the orthodontic device case includes a divider for separating different pieces of the orthodontic device.

19. The attachable orthodontic device case of claim 18, further comprising:
an ultraviolet light located within the interior volume of the orthodontic device case, wherein said light is activated when the case is closed.

20. An attachable orthodontic device case comprising:
an orthodontic device case having an interior volume configured for holding an orthodontic device;
the orthodontic device case comprising an opening configured for insertion of the orthodontic device; and
a flat surface on a rear of the orthodontic device case and a magnet on said flat surface for coupling to a rear of a mobile computing device.
